(19) United States
(12) Patent Application Publication
(10) Pub. No.: US 2025/0332366 A1
(43) Pub. Date: Oct. 30, 2025

(54) RESPIRATORY MASK

(71) Applicant: Neville Cameron, Grayson, GA (US)

(72) Inventor: Neville Cameron, Grayson, GA (US)

(21) Appl. No.: 18/651,344

(22) Filed: Apr. 30, 2024

(51) Int. Cl. A61M 16/06; A61M 16/00

(52) U.S. Cl. CPC A61M 16/0605; A61M 16/0003; A61M 2205/11; A61M 2205/60; A61M 2209/00; A61M 2210/0606

(57) ABSTRACT

The respiratory mask is formed from a suitable plastic material that is non-permeable to both ambient air and typical respiratory gases, and has sufficient flexibility so as to conform to a patient's face. The respiratory mask includes a mask body that is generally concave, so as to define a cavity adapted to fit over the mouth and nose of a patient, a gas inlet port, and a rotating dial with two instrument access ports. Each instrument access port provides access into the cavity defined by the mask body for an instrument and can be used to guide the instrument into the mouth or nose of the patient. The rotating dial is configured to rotate, allowing each of the instrument access ports to be selectively aligned with the mouth or nose of the patient.

### TECHNICAL FIELD

This disclosure relates generally to a respiratory mask, and specifically to a respiratory mask equipped with repositionable instrument access ports.

### BACKGROUND

There is currently a need in the medical profession for a respiratory mask that would allow medical personnel to perform diagnostic and therapeutic procedures while concomitantly maintaining adequate oxygenation. Furthermore, it would be advantageous if any instrument access ports of the respiratory mask could be repositioned to facilitate inserting an instrument into the oral or nasal passages of a patient, depending on the diagnostic and/or therapeutic procedure being performed.

Accordingly, needs exist for the respiratory mask disclosed herein. It is to the provision of a respiratory mask configured to address these and other needs that the present invention is primarily directed.

### SUMMARY OF THE INVENTION

It is to be understood that this summary is not an extensive overview of the disclosure. This summary is exemplary and not restrictive, and it is intended neither to identify key or critical elements of the disclosure nor delineate the scope thereof. The sole purpose of this summary is to explain and exemplify certain concepts of the disclosure as an introduction to the following complete and extensive detailed description.

Disclosed is a respiratory mask formed from a suitable plastic material that is non-permeable to both ambient air and typical respiratory gases, and has sufficient flexibility so as to conform to a patient's face. An example respiratory mask comprises a mask body that is generally concave, so as to define a cavity adapted to fit over the mouth and nose of a patient, a gas inlet port, and a rotating dial with two instrument access ports. Each instrument access port provides access for an instrument and can be used to guide the instrument into the mouth or nose of the patient. The rotating dial is configured to rotate, allowing each of the instrument access ports to be selectively aligned with the mouth or nose of the patient.

Also disclosed is a sanitary sleeve adapted for use with an instrument access port in a respiratory mask, such as the respiratory mask described above. The sanitary sleeve is used in conjunction with a suitably configured insertion end of an instrument meant for insertion into the mouth and/or nose of a patient. The sanitary sleeve is configured to prevent the insertion end of an instrument from coming into contact with the respiratory mask during insertion and removal.

An example sanitary sleeve comprises a cylindrical body with an annular ledge on one end, a guide opening for an insertion end of an instrument, and an indexing tab configured to releasably engage with an annular groove in the insertion end of the instrument. The cylindrical body is configured to fit within an instrument access port of a respiratory mask, and the cylindrical body defines an interior space configured to collect fluid. The annular ledge is configured to prevent over-insertion of the sanitary sleeve into the instrument access port. The guide opening is configured to allow the insertion end of the instrument to be advanced into, and retracted from, an oral or nasal orifice of a patient.

### BRIEF DESCRIPTION OF THE DRAWINGS

FIG. 1 is an isometric view of a respiratory mask according to the principles of the present disclosure being worn.

FIG. 2 is an isometric view of the respiratory mask shown in FIG. 1.

FIG. 3 is a front elevational view of the respiratory mask shown in FIG. 2.

FIG. 4 is a side elevational view of the respiratory mask shown in FIG. 2.

FIG. 5 is bottom plan view of the respiratory mask shown in FIG. 2.

FIG. 6 is a rear elevational view of the respiratory mask shown in FIG. 2.

FIG. 7 is a top plan view of the respiratory mask shown in FIG. 2.

FIG. 8 is a cutaway view of the respiratory mask taken along line 8-8 shown in FIG. 7.

FIG. 9 is an exploded isometric view of the respiratory mask shown in FIG. 2.

FIG. 10 is an isometric view of the sanitary sleeve and insertion end of an instrument shown in FIG. 2.

FIG. 11 a side elevational view of the sanitary sleeve and insertion end of an instrument shown in FIG. 10.

FIG. 12 is a side-cutaway view of the sanitary sleeve and insertion end taken along line 12-12 shown in FIG. 11.

FIG. 13 is an isometric view of the sanitary sleeve shown in FIG. 10.

FIG. 14 is a top plan view of the sanitary sleeve shown in FIG. 13.

FIG. 15 is an isometric view of the insertion end shown in FIG. 10.

Like reference numerals refer to corresponding parts throughout the several views of the drawings.

### DETAILED DESCRIPTION

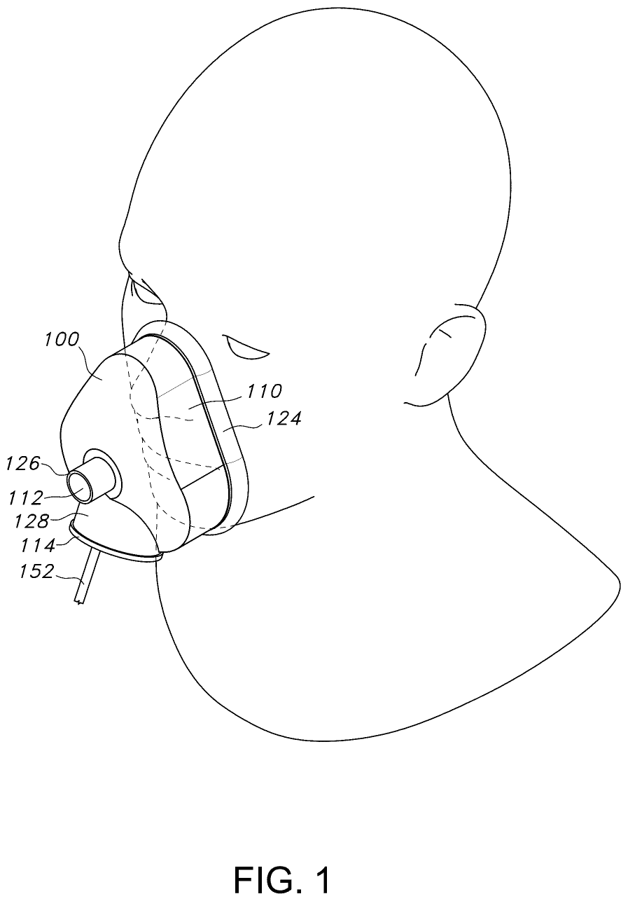

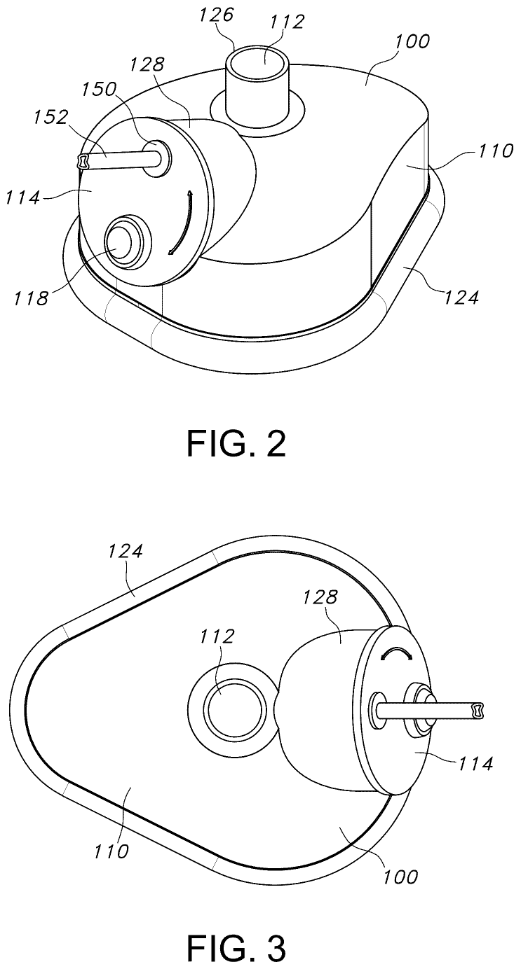

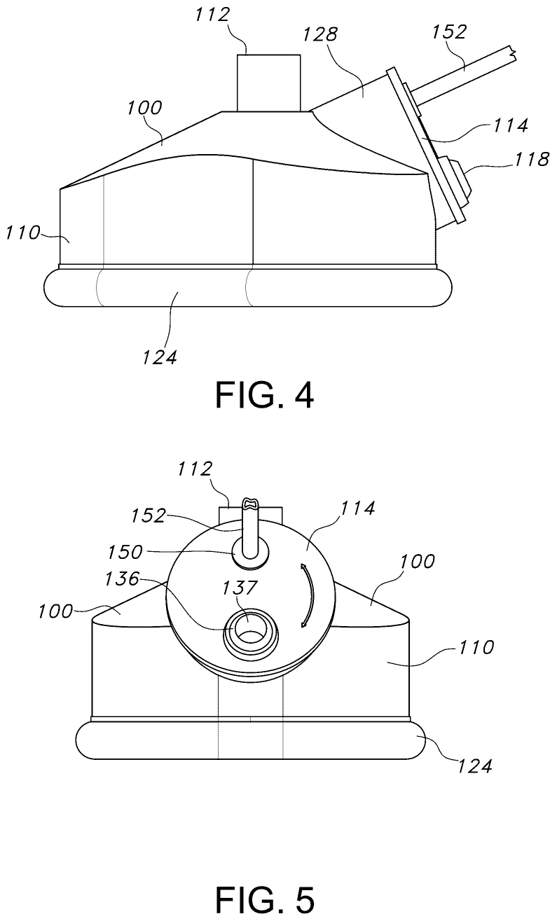

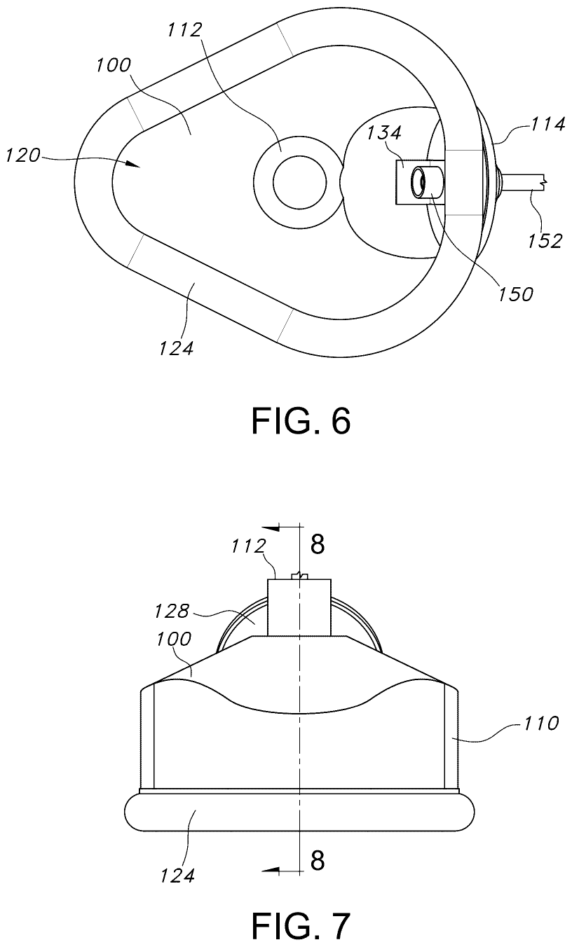

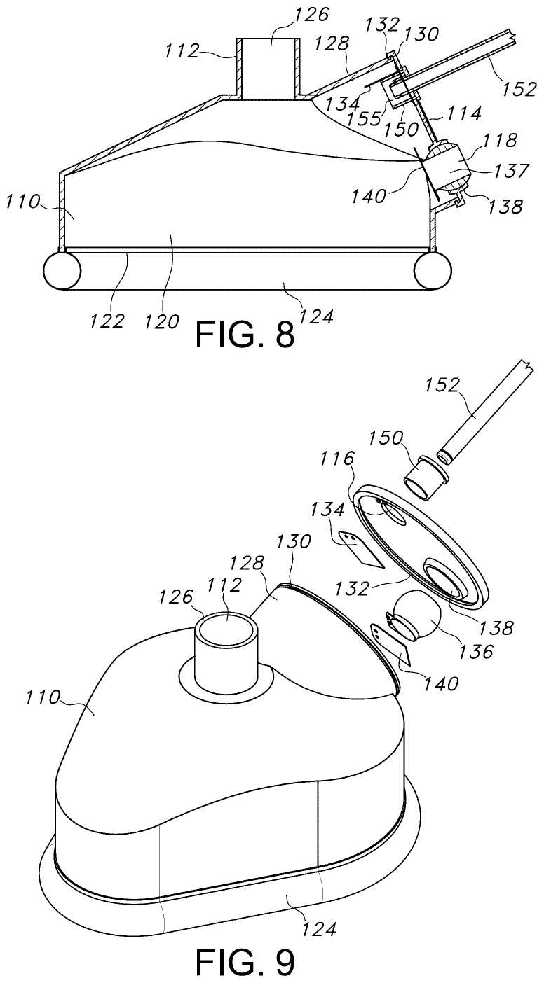

FIGS. 1-9 illustrate a respiratory mask 100 according to the principles of the present disclosure. The respiratory mask 100 is formed from a suitable plastic material that is non-permeable to both ambient air and typical respiratory gases, and has sufficient flexibility so as to conform to a patient's face. As shown in FIG. 1, the respiratory mask 100 is configured to cover the mouth and nose of a patient and includes a gas inlet 112 port and a pair of instrument access ports (116, 118), each of which provides access for an instrument and can be used to guide the instrument into the oral or nasal orifice of the patient.

As shown best in FIGS. 1-9, the respiratory mask 100 comprises a mask body 110 that is configured to cover the mouth and nose of a patient. The mask body 110 includes a gas inlet port 112 and a rotating dial 114 with a pair of instrument access ports (116, 118). The dial 114 is configured to rotate, allowing each of the instrument access ports (116, 118) to be selectively aligned with the mouth or nose of the patient.

The mask body 110 is generally concave, so as to define a cavity 120 via which supplemental oxygen or pressurized air is delivered to the patient. The cavity 120 is adapted to fit over the mouth and the nose of a patient. The surrounding peripheral edge 122 of the mask body 110 is preferably contoured so as to substantially seal against the surrounding facial tissue of the patient, thereby establishing the cavity 120. In some implementations, the peripheral edge of the mask body 110 may have an enhanced seal 124, such as foam, flexible plastic, or a rubberized material, for improved performance.

The gas inlet port 112 of the mask body 110 is configured for connection to an oxygen source, for example, an oxygen tank or a self-inflating bag. The gas inlet port 112 is a cylindrical tube that extends outwardly from the mask body 110. The free end 126 of the gas inlet port 112 is disposed outside of the mask body 110. In use, a supply of inhalation gas, such as oxygen or pressurized air, is connected to the free end 126 of the gas inlet port 112 via a supply tube so as to supply the inhalation gas to the cavity 120 of the respiratory mask 100 and thereby the airways of the patient.

The rotating dial 114 is secured to a connector port 128, located below the gas inlet port 112 of the mask body 110. However, the rotating dial 114 could be positioned elsewhere on the mask body 110. The connector port 128 extends outwardly and downwardly from the mask body 110, and includes an outwardly extending annular lip 130 that fits under a corresponding retention flange 132 on the backside of the rotating dial 114. In this way, the rotating dial 114 is both securely attached to the connector port 128 and able to rotate. The annular retention flange 132, formed on the backside of the rotating dial 114, is configured to work in conjunction with the annular lip 130 on the end of the connector port 128. The inside diameter of the annular retention flange 132 is less than the outside diameter of the annular lip 130 on the end of the connector port 128 such that when the rotating dial 114 is pressed against the end of the connector port 128, the pliant material of the mask body 110 deforms and permits a snap fit of the rotating dial 114 onto the end of the connector port 128. This snap fit prevents gas from escaping between the rotating dial 114 and connector port 128, while still allowing the rotating dial 114 to be turned.

The instrument access ports (116, 118) can be used for inserting instruments into the mouth and/or nose of the patient. Each of the instrument access ports (116, 118) is configured to facilitate easy access for instruments, such as thermometers, suction tubes, and feeding tubes. One of the instrument access ports is a fixed access port 116, and the other is a flexible access port 118.

The fixed access port 116, an opening in the rotating dial 114, allows access into the cavity 120 defined by the mask body 110. A flap valve 134, configured to close the fixed access port 116 when not in use, prevents expiratory flow from escaping through the fixed access port 114, but allows the passage of an instrument as shown in FIG. 8. The flap valve 134 comprises a layer of elastomeric material, secured at one end to an interior portion of the rotating dial 114 adjacent to the fixed access port 116.

The flexible access port 118 includes a fitting 136 configured to allow access into the cavity defined by the mask body 110. The fitting 136 includes an opening 137 that extends therethrough and is configured to pivot, allowing for an instrument to be more precisely guided into the mouth or nose of a patient wearing the respiratory mask 100. The fitting 136 has a rounded exterior and is seated in a complementary socket 138 on the rotating dial 114, forming a ball-and-socket arrangement that allows the fitting 136 to pivot. A flap valve 140, configured to close the flexible access port 118 when not in use, prevents expiratory flow from escaping through the flexible access port 118, but allows the passage of an instrument. The flap valve 140 comprises a layer of elastomeric material, secured at one end to an interior portion of the rotating dial 114 adjacent to the flexible access port 118.

While the rotating dial 114 is shown and described as having both a fixed access port 116 and flexible access port 118, it should be understood that the rotating dial 114 could be configured to include either a pair of fixed access ports 116 or a pair of flexible access ports 118. Also, it should be understood that a rotating dial 114 could be configured to include only a single instrument access port or more than two instrument access ports.

Although not shown, the respiratory mask 110 could be configured to include a flexible strap, allowing it to be attached to a patient.

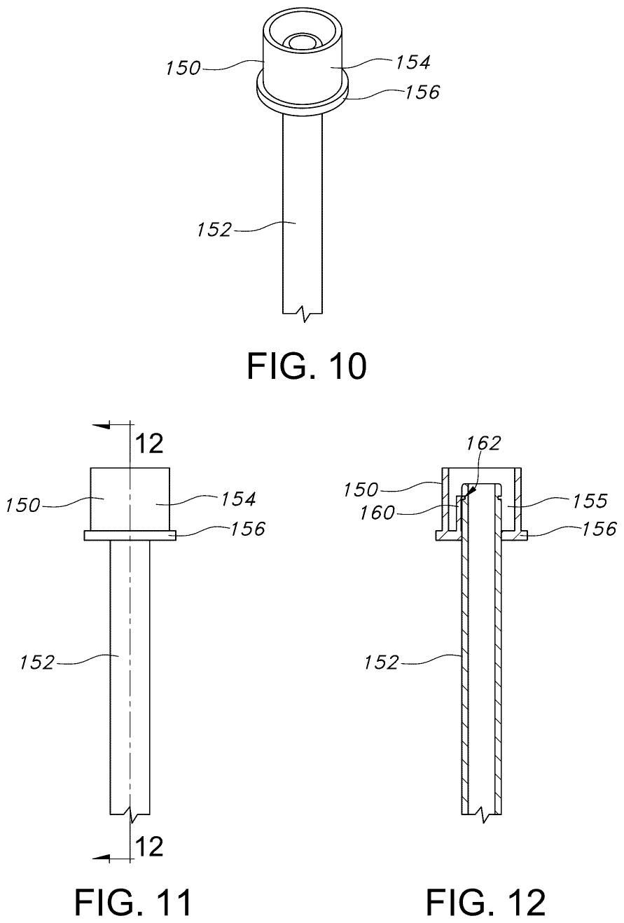

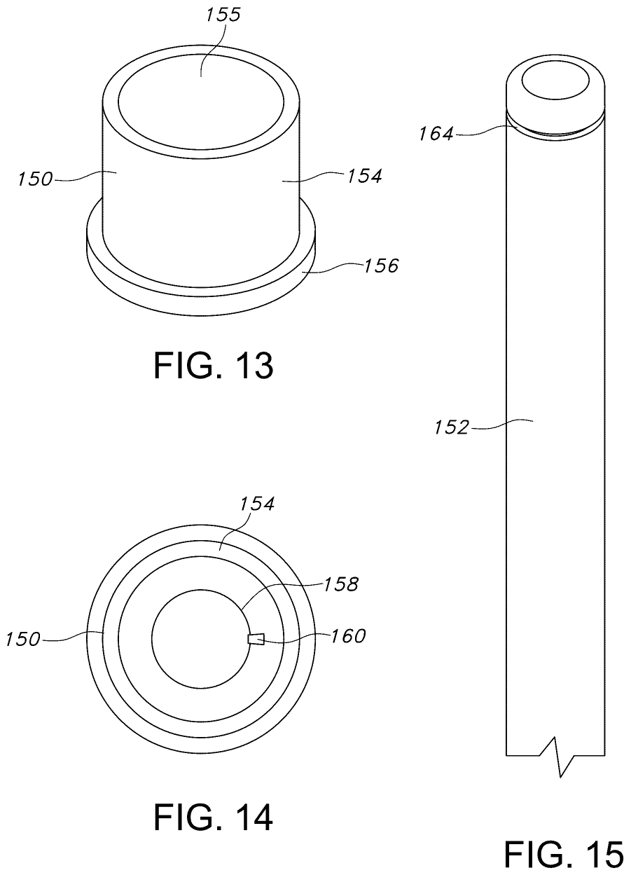

FIGS. 10-12 illustrate a sanitary sleeve 150 in conjunction with an example insertion tip 152 of an instrument. An end of the insertion tip 152 is illustrated with a break line to indicate that the remainder of the instrument is not shown in the drawings. The sanitary sleeve 150 is configured to fit within the fixed access port 116 of the respiratory mask 100 (see, e.g., FIG. 8). However, the sanitary sleeve 150 could be adapted for use with other instrument access ports. The sanitary sleeve 150 is used in conjunction with a suitably configured insertion end 152 of an instrument meant for insertion into the mouth and/or nose of a patient. The sanitary sleeve 150 is configured to prevent the insertion end 152 of an instrument from coming into contact with the mask body 110 during insertion and removal. The sanitary sleeve 150 is also configured to prevent the insertion end 152 of an instrument from coming into contact with the flap valve 134 during use.

The sanitary sleeve 150 comprises a cylindrical body 154 with an annular ledge 156 at one end, a guide opening 158 for the insertion end 152 of an instrument, and an indexing tab 160 configured to engage with an annular groove 164 in the insertion end 152 of the instrument.

The cylindrical body 154 of the sanitary sleeve 150 is configured to fit within both the fixed access port 116 and the flexible access port 118. The cylindrical body 154 defines an interior space 155 configured to collect fluid. This fluid may accumulate on the insertion end 152 during use, as well as inside the respiratory mask 100 around the fixed access port 116 when the sanitary sleeve 150 is used with an instrument that has a compatible insertion tip 152. The annular ledge 156, extending radially from the cylindrical body 154, is configured to prevent over-insertion of the sanitary sleeve 150.

The guide opening 158 in the sanitary sleeve 150 is configured to allow the insertion end 152 of an instrument to be advanced into, and retracted from, the oral or nasal orifice of the patient, corresponding to the procedure to be performed.

The indexing tab 160, positioned within the interior space 155 defined by the cylindrical body 154 of the sanitary sleeve 150, includes a projecting lip 162. The indexing tab 160 is flexible and configured to press the projecting lip 162 against the insertion end 152 of an instrument, causing the projecting lip 162 to engage with the annular groove 164 on the insertion end 152 when the instrument is sufficiently retracted from the sanitary sleeve 150 (see, e.g., FIG. 12). While the indexing tab 160 is engaged with the annular groove 164, the tip of the insertion end 152 is recessed relative to the end of the sanitary sleeve 150. In this way, the sanitary sleeve 150 is configured to prevent the insertion end 152 of an instrument from coming into contact with the mask body 110 during insertion and removal. While the indexing tab 160 is engaged with the annular groove 164 on the insertion end 152 of a suitably configured instrument, the instrument can be used to insert and withdraw the sanitary sleeve 150 from the fixed access port 116 of the respiratory mask 100. The projecting lip 162 of the indexing tab 160 is configured to disengage from the annular groove 164 on the insertion end 152 when the instrument is pushed into the mask cavity 120.

Although not shown, the sanitary sleeve 150 can be configured to accommodate a variety of instruments, such as spittoons, suction devices, thermometers, and other medical instruments. For compatibility, these instruments will need to include an insertion end configured to pass through the guide opening 158 in the sanitary sleeve 150. The insertion end will also need to have a suitably configured annular groove 154.

The foregoing description of the invention is intended to be illustrative; it is not intended to be exhaustive or to limit the claims to the precise forms disclosed. Those skilled in the relevant art can appreciate that many modifications and variations are possible in light of the foregoing description and associated drawings.

Reference throughout this specification to an “embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A respiratory mask comprising:
a mask body that is generally concave, so as to define a cavity adapted to fit over the mouth and nose of a patient;
a gas inlet port; and
a rotating dial with two instrument access ports, each of which provides access into the cavity for an instrument and can be used to guide the instrument into the mouth or nose of the patient;
wherein:
the rotating dial is configured to rotate, allowing each of the instrument access ports to be selectively aligned with the mouth or nose of the patient.

2. The respiratory mask of claim 1, wherein the mask body includes a connector port, and the rotating dial is secured to the connector port.

3. The respiratory mask of claim 2, wherein the connector port includes an outwardly extending annular lip that fits under a corresponding annular retention flange on a backside of the rotating dial.

4. The respiratory mask of claim 3, wherein an inside diameter of the annular retention flange is less than an outside diameter of the outwardly extending annular lip on the connector port.

5. The respiratory mask of claim 1, wherein at least one of the two instrument access ports is an opening in the rotating dial, and the at least one instrument access port includes a flap valve configured to close the opening when not in use.

6. The respiratory mask of claim 1, wherein at least one of the two instrument access ports includes a rounded fitting, seated in a socket in the rotating dial and configured to pivot; the at least one instrument access port also includes a flap valve configured to close the at least one instrument access port when not in use.

7. A sanitary sleeve used in conjunction with an instrument, meant for insertion into the mouth or nose of a patient, having an insertion end with an annular groove, the sanitary sleeve comprising:
a cylindrical body with an annular ledge on one end;
a guide opening for the insertion end of the instrument; and
an indexing tab configured to releasably engage with the annular groove in the insertion end of the instrument;
wherein:
the cylindrical body is configured to fit within an instrument access port of a respiratory mask, and the cylindrical body defines an interior space configured to collect fluid;
the annular ledge is configured to prevent over-insertion of the sanitary sleeve into the instrument access port; and
the guide opening is configured to allow the insertion end of the instrument to be advanced into an oral or nasal orifice of a patient.

8. The sanitary sleeve of claim 7, wherein the indexing tab, positioned within the interior space defined by the cylindrical body, includes a projecting lip configured to releasably engage with the annular groove in the insertion end of the instrument.
